(19) United States
(12) Patent Application Publication
(10) Pub. No.: US 2017/0340220 A1
(43) Pub. Date: Nov. 30, 2017

(54) PHYSIOLOGICAL DETECTION METHOD AND DEVICE THEREOF

(71) Applicant: Leadtek Research Inc., New Taipei City (TW)

(72) Inventors: Po-Chun Hsu, New Taipei City (TW); Cheng-Jun Chuang, New Taipei City (TW); Mike Chang, New Taipei City (TW); Kuo-Hung Cheng, New Taipei City (TW); Jason Yang, Taipei City (TW); Yu-Hsiang Lin, New Taipei City (TW); Chao-Jung Yu, Taoyuan City (TW)

(73) Assignee: Leadtek Research Inc., New Taipei City (TW)

(21) Appl. No.: 15/278,014

(22) Filed: Sep. 27, 2016

(30) Foreign Application Priority Data
May 30, 2016 (TW) 105116804

(51) Int. Cl. A61B 5/024; A61B 5/0255; A61B 5/00

(52) U.S. Cl. CPC A61B 5/02416; A61B 5/742; A61B 5/0255; A61B 5/7225

(57) ABSTRACT

A physiological detection method includes the following steps. A detection portion of a human body is detected to obtain a detection signal. Then, the detection signal is processed to output a digital physiological signal. The digital physiological signal is received to calculate and obtain first information and second information related to feature points thereof. Then, a ratio of the second information to the first information is calculated to obtain a physiological condition index. The digital physiological signal includes pulse waves generated according to a time sequence. The feature points of the digital physiological signal include a wave pulse peak and a foot point located at a forepart of the rising edge of the wave. In addition, a physiological detection device is also introduced.

### CROSS-REFERENCE TO RELATED APPLICATION

This application claims the priority benefit of Taiwan application serial no. 105116804, filed on May 30, 2016. The entirety of the above-mentioned patent application is hereby incorporated by reference herein and made a part of this specification.

### BACKGROUND

### Field of the Invention

The invention is directed to a physiological detection method and more particularly, to a physiological detection method for detecting a body circulation condition.

### Description of Related Art

Cardiovascular diseases have become one of the main causes of death around the world. Thus, various methods for detecting cardiovascular circulation of human bodies as well as research and development thereof have drawn attention more widely. Among currently available detection methods, a method of measuring peripheral blood circulation of human bodies by using a photoplethysmography signal emitted by a photoplethysmograph (PPG) has been gradually viewed important. The PPG is configured to capture an optical volume pulse of blood of a measurement portion in a human body and further calculate a physiological condition index according to the captured optical volume pulse by using a calculator.

Specifically, the calculator is configured to calculate a physiological condition index according to information related to feature points of an optical volume pulse signal of the human measurement portion. FIG. 1 is a waveform chart of a volume pulse of a digital physiological signal of the related art. Referring to FIG. 1, in the calculation method of a physiological condition index of the related art, a vascular reflection index is calculated according to a ratio of a height difference a between a trough point d3 and a pulse peak d1 (i.e., a systolic wave pulse peak) to a height difference b between the trough point d3 and a diastolic wave peak d2. In addition, in the calculation method of the related art, a ratio of a subject's height to a time difference Td between the systolic wave pulse peak d1 and the diastolic wave peak d2 is calculated to serve as a vascular stiffness index.

However, the calculation method of the physiological condition index has defects. To be detailed, an optical volume pulse signal of a normal subject has a pulse wave with a transient rebound and rise during the process of descending, which is the above-mentioned diastolic wave. However, as for a subject who is in a poor health condition or aged, an optical volume pulse signal obtained by detecting his or her detection portion may not have a diastolic wave, or a position of the diastolic wave peak may be unobvious, and as a result, a physiological condition index of the subject cannot be obtained effectively by utilizing the aforementioned calculation method. Thus, the detection and calculation method of the physiological condition index described above is not applicable to all subjects to be detected. Accordingly, how to provide a physiological detection method that is correctly and simply applicable for detection results of all subjects has become a major issue to technicians of the art.

### SUMMARY

The invention provides a physiological detection method capable of calculating a physiological condition index according to feature points of a digital physiological signal to assess a peripheral circulation condition of a human body in a simple way according to the physiological condition index.

The invention provides a physiological detection device capable of detecting and assessing a peripheral circulation condition of a human body in a non-invasive manner.

A physiological detection method of the invention includes the following steps. A detection portion of a human body is detected to obtain a detection signal. Then, the detection signal is processed to output a digital physiological signal. The digital physiological signal is received to obtain first information and second information related to feature points of the digital physiological signal, and a ratio of the second information to the first information is calculated to obtain a physiological condition index. The digital physiological signal includes a plurality of pulse waves generated according to a time sequence, and the feature points of the digital physiological signal include a pulse peak of each pulse wave and a foot point located at a forepart of a rising edge of each pulse wave.

A physiological detection device of the invention includes a detector, a signal processor and a calculation module. The detector is adapted to detect a detection portion of a human body to obtain a detection signal. The signal processor receives and processes the detection signal to output a digital physiological signal. The calculation module receives the digital physiological signal and obtains first information and second information related to a plurality of feature points of the digital physiological signal. The calculation module calculates a ratio of the second information to the first information to obtain a physiological condition index. The digital physiological signal includes a plurality of pulse waves generated according to a time sequence, and the feature points of the digital physiological signal include a pulse peak of each pulse wave and a foot point located at a forepart of a rising edge of each pulse wave.

In an embodiment of the invention, the first information is an integrated area of the pulse wave between the foot point and the pulse peak with respect to a time axis, and the second information is an integrated area of the pulse wave between two adjacent foot points with respect to the time axis.

In an embodiment of the invention, the first information is a time difference between the foot point and the pulse peak, and the second info′ nation is a time difference between two adjacent foot points.

In an embodiment of the invention, the step of processing the detection signal to output the digital physiological signal includes: filtering, amplifying the detection signal, and converting the detection signal into the digital physiological signal.

In an embodiment of the invention, the step of calculating the information related to the feature points to obtain the physiological condition index includes: normalizing the digital physiological signal, and calculating the physiological condition index according to the first information and the second information related to the feature points of the normalized digital physiological signal.

In an embodiment of the invention, the detector is a photoplethysmograph (PPG). The PPG includes an optical emitter and an optical receiver. The optical emitter is configured to emit a light passing through the detection portion of the human body. The optical receiver is configured to receive the light passing through the detection portion to obtain the detection signal.

In an embodiment of the invention, the signal processor includes a filter, an amplifier and an analog-to-digital converter. The filter is configured to filter the detection signal. The amplifier is configured to amplify the detection signal. The analog-to-digital converter is configured to convert the detection signal into the digital physiological signal.

In an embodiment of the invention, the calculation module includes a normalization processor and a physiological condition index calculator. The normalization processor is configured to normalize the digital physiological signal. The physiological condition index calculator is configured to calculate the physiological condition index according to the feature points of the normalized digital physiological signal.

To sum up, in the physiological detection method provided by the embodiments of the invention, the detecting device detects the detection portion of the human body to obtain the detection signal with respect to a physiological condition of the detection portion. In addition, the signal processor further processes the detection signal to output the digital physiological signal. Furthermore, the calculation module calculates a plurality of feature points according to the digital physiological signal, and then, calculates the physiological condition index according to feature points of the digital physiological signal. In the plurality of embodiments of the invention, a human physiological condition can be assessed according to the physiological condition index obtained by the method and the device described above in a simple way for assessing, such that the time, process, equipment and related cost required for the physiological detection can be reduced.

To make the above features and advantages of the invention more comprehensible, embodiments accompanied with drawings are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a waveform chart of a volume pulse of a digital physiological signal of the related art.

FIG. 2 is a schematic block view of a physiological detection device according to an embodiment of the invention.

FIG. 3A to FIG. 3C are waveform charts of volume pulses of pulse waves of a digital physiological signal of the physiological detection device depicted in FIG. 2.

FIG. 4 is a flowchart illustrating a physiological detection method according to an embodiment of the invention.

FIG. 5 is a flowchart of the signal processing step of the physiological detection method depicted in FIG. 4.

FIG. 6 is a flowchart of the step of calculating the physiological condition index of the physiological detection method depicted in FIG. 4.

### DESCRIPTION OF EMBODIMENTS

In the embodiments provided below, the same or similar symbols represent components or devices having the same or similar functions, wherein shapes, sizes and ratios of the devices in the drawings are merely schematically illustrated and construe no limitations to the invention. Additionally, although several technical features may be simultaneously described in any one of the embodiments below, it does not indicate that all the technical features have to be simultaneously implemented in the embodiment.

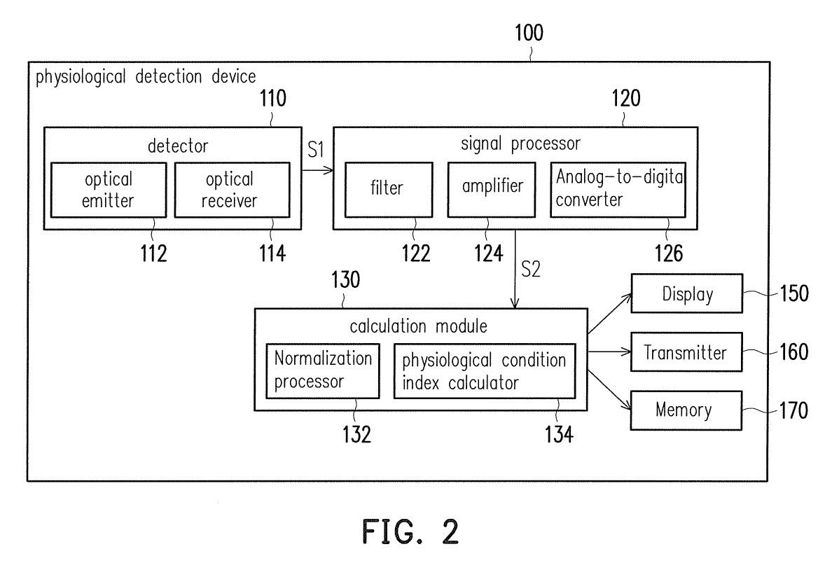

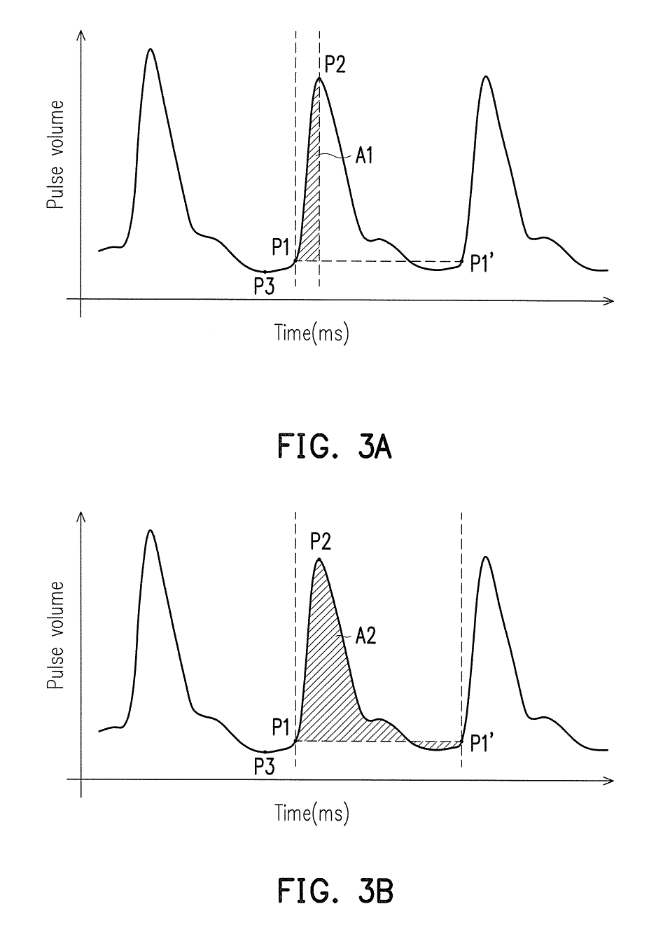

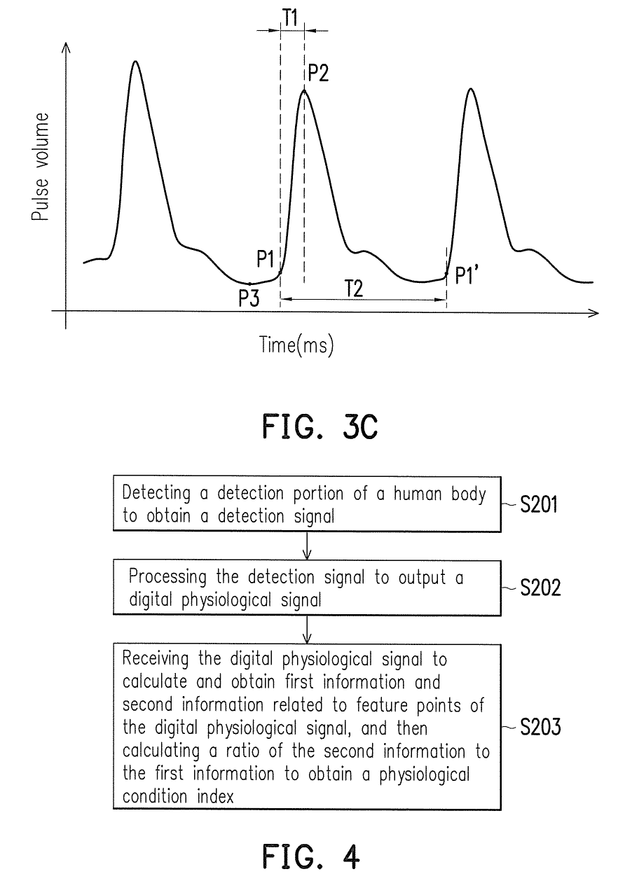

FIG. 2 is a schematic block view of a physiological detection device according to an embodiment of the invention. FIG. 3A to FIG. 3C are waveform charts of volume pulses of pulse waves of a digital physiological signal of the physiological detection device depicted in FIG. 2. Referring to FIG. 2 and FIG. 3, in the present embodiment, a physiological detection device 100 includes a detector 110, a signal processor 120 and a calculation module 130. The detector 110 is, for example, a photoplethysmograph (PPG). The detector 110 is configured to detect and determine a physiological condition of a detection portion of a human body according to a light with a specific wavelength emitted and received by the detector 110 and an amount of spectral energy of the light which is absorbed. For example, the detection portion of the human body may be a peripheral portion, e.g., a human earlobe, finger or toe. In the present embodiment, the detector 110 includes one set of optical emitter 112 and optical receiver 114 or more, and the type of the optical emitter 112 and the optical receiver 114 may be transmissive or reflective. Thus, a light emitted by the optical emitter 112 penetrates through the human detection portion or is reflected by the detection portion and then, is received by the corresponding optical receiver 114.

The optical emitter 112 and the optical receiver 114 of the present embodiment are, for example, an infrared optical emitter and an infrared optical receiver, and a wavelength of the light emitted by the optical emitter and received by the optical receiver falls within a range between 760 nm and 1 μm. However, the present embodiment is not limited thereto. According to a detection demand of the physiological detection device 100, the light emitted by the optical emitter 112 and received by the optical receiver 114 may also be a green light (with a wavelength falling a range between 495 nm and 570 nm), a red light (with a wavelength falling a range between 620 nm and 750 nm) or a light of other types or having other wavelength ranges.

To be detailed, the detector 110 of the physiological detection device 100 is configured to obtain a detection signal S1, and the detection signal S1 of the present embodiment may be a PPG signal emitted by the PPG. In the present embodiment, the optical receiver 114 of the detector 110 has a light sensing element (not shown), and the light sensing element may be configured to receive the light passing through or reflected from the human detection portion. Thus, the detector 110 estimates a blood volume variation in a vessel by detecting, for example, an amount of spectral energy absorbed by hemoglobin of the blood in the detection portion. It is to be mentioned that a concentration of hemoglobin in human blood may be approximately considered as constant. Thus, in a general condition, an amount of hemoglobin detected in a vessel may be employed to estimate the blood volume variation in the vessel, so as to obtain the detection signal S1.

When the light passes through the human vessel, the absorbed amount of the spectral energy of the light varies with pulsation of the heart. Specifically, a unit area of the vascular wall expands and contracts as the heart pulsates and the blood flows through. Thus, the light passing through the vessel generates a quasi-periodic variation along with the expansion and contraction of the vessel and a variation in an amount of blood perfusion in the vessel, so as to generate the quasi-periodic detection signal S1.

Generally speaking, when a human heart contracts, the blood is pumped into the artery, and in this circumstance, the absorbed amount of the spectral energy of the light increases along with the increase of the blood volume of the vessel, so as to obtain the detection signal S1 in a greater degree. Thus, the degree of the detection signal S1 is proportional to the blood volume (blood perfusion) of the vessel of the human detection portion.

Referring to FIG. 2 again, the signal processor 120 is coupled to the detector 110 to receive the detection signal S1 generated by the detector 110. The signal processor 120 of the present embodiment includes a filter 122, an amplifier 124 and an analog-to-digital converter 126. In the present embodiment, the filter 122 performs a bandpass filtering operation on the received detection signal S1, and a filtering frequency falls within a range between 0.5 Hz and 5 Hz. The filtering range of the filter 122 may be adaptively changed according to different detection demands.

The amplifier 124 of the signal processor 120 is configured to automatically gain the detection signal S1 to an adaptive size. In addition, the analog-to-digital converter 126 is configured to convert the detection signal S1 which is amplified but still an analog signal into a digital physiological signal S2 for subsequent signal processing and related computation.

In the present embodiment, as described above, the detection signal S1 may be first amplified by the amplifier 124, and then the detection signal S1 which is originally an analog signal may be converted into the digital physiological signal S2 by the analog-to-digital converter 126. Alternatively, the detection signal S1 may be first converted into the digital physiological signal S2 by the analog-to-digital converter 126 and then amplified by the amplifier 124.

The calculation module 130 is coupled to the signal processor 120 and configured to calculate the digital physiological signal S2 to obtain information related to feature points of the digital physiological signal S2. Referring to FIG. 3A, in the present embodiment, the blood is periodically perfused from the heart to the vessel in correspondence to the pulsation of the heart, the digital physiological signal S2 has a plurality of pulse waves generated according to a time sequence, and a size of each pulse wave corresponds to the volume of the blood entering the vessel. Referring to FIG. 3A, the feature points of the digital physiological signal S2 include a pulse peak P2 of each pulse wave, a trough point P3 of each pulse wave and a foot point P1 located at a forepart of a rising edge of each pulse wave. In the present embodiment, the foot point P1 of each pulse wave indicates a pressure of a vascular wall and an intravascular blood volume when the human heart ends the diastole and starts to contract.

The pulse peak P2 of each pulse wave is a peak of each pulse wave, and the pulse peak P2 indicates a maximum pulse wave amplitude induced by the blood injected to the vessel from the heart when the heart contracts. In the present embodiment, a rising band from the foot point P1 to the pulse peak P2 represents a state of rapid expansion of the vascular wall as the intravascular blood volume in the artery increases rapidly when the blood is rapidly injected from the heart ventricle. In addition, a declining band from the pulse peak P2 represents a state that the intravascular blood volume of the artery gradually decreases, and the vascular wall gradually returns to the condition before expansion. It is to be mentioned that the rising amplitude of the pulse waveform of the digital physiological signal S2 from the foot point P1 to the pulse peak P2 is influenced by a quantity of the blood output from the heart, arterial resistance, elasticity of the vascular wall and a speed of the heart ventricle injecting the blood. Additionally, it is well known to persons skilled in the art that as the rising amplitude of the pulse wave between the foot point P1 and the pulse peak P2 is becomes greater, a time difference from the foot point P1 to the pulse peak P2 is shorter, which represents a better perfusion condition of the blood in the vessel. Namely, if the vessel is capable of expanding in a shorter time, it represents that the vascular wall has a smaller degree of stiffness and better elasticity.

In the present embodiment, the calculation module 130 includes a normalization processor 132 and a physiological condition index calculator 134. After the calculation module 130 calculates and obtains the feature points of the digital physiological signal S2, the calculation module 130 further normalizes the digital physiological signal S2 by using the normalization processor 132, such that the digital physiological signal S2 returns to its original size before being amplified by the amplifier 124. Then, the physiological condition index calculator 134 of the calculation module 130 calculates the physiological condition index according to first information and the second information related to the feature points of the digital physiological signal S2.

To be detailed, referring to FIG. 3A and FIG. 3B, the horizontal axis of each of the pulse waveform charts of FIG. 3A and FIG. 3B represents a time axis, of which the unit is millisecond (ms), and the vertical axis corresponds to a size of each pulse wave volume of the digital physiological signal S2. In the present embodiment, the information related to the feature points includes the first information and the second information. The first information is an integrated area A1 of the pulse wave between the foot point P1 and the pulse peak P2 with respect to the time axis as illustrated in FIG. 3A, and the second information is an integrated area A2 of the pulse wave between two adjacent foot points P1 and PP (which also refers to a complete heartbeat period) with respect to the time axis as illustrated in FIG. 3B. In addition, the physiological condition index calculator 134 calculates a ratio of the first information to the second information, i.e., a ratio of the integrated area A2 to the integrated area A1, so as to obtain a corresponding physiological condition index and accordingly assess the perfusion condition of the blood in the vessel and a blood circulation condition of the body. Besides, in the present embodiment, the areas may be calculated by utilizing various means of calculating a leftward shift of an amplitude which are commonly used in the computer science field for reducing the amount of computation.

Referring to FIG. 3C, in another embodiment, the first information related the feature points of each pulse wave may be a time difference T1 between the foot point P1 and the pulse peak P2 as illustrated in FIG. 3C, and the second information may be a time difference T2 between two adjacent foot points P1 as illustrated in FIG. 3C. The physiological condition index calculator 134 of the calculation module 130 may also calculate the ratio of the first information to the second information, i.e., a ratio of the time difference T1 to the time difference T2, so as to obtain the corresponding physiological condition index and accordingly assess the perfusion condition of the blood in the vessel and the blood circulation condition of the body.

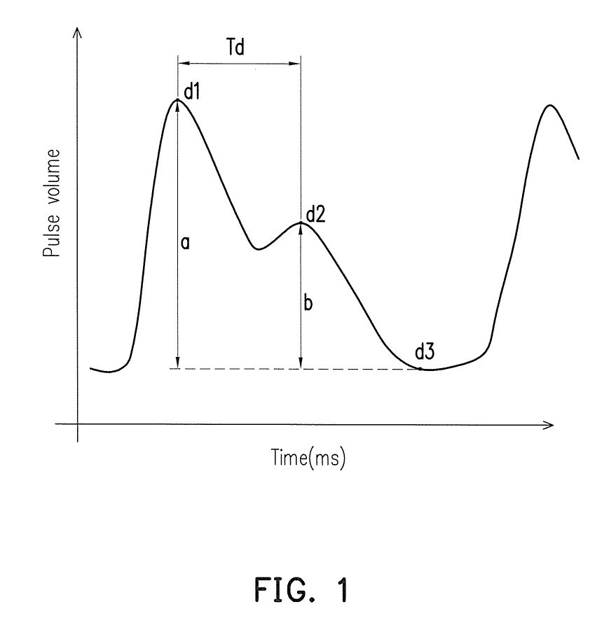

In comparison with the content of the related art as illustrated in FIG. 1, the calculation of the physiological condition index does not reply on the diastolic pulse wave of the digital physiological signal S2 of the subject to obtain the second information in the present embodiment. Specially, the pulse wave of the digital physiological signal S2 measured from the subject who is aged or in a poor health condition usually lacks the diastolic wave, or the position of the diastolic wave peak is unobvious, such that due to the failure in effectively obtaining the second information from the pulse wave, the calculation module 130 is incapable of calculating the ratio of the second information to the first information for obtaining the physiological condition index.

The second information of the present embodiment is directly captured from the pulse wave between the two foot points P1 and P1′, i.e., the second information is directly captured from the pulse wave of a complete period. Thus, in the calculation of the physiological condition index of the present embodiment, besides from the pulse wave between the two foot points P1 and P1′, the second information may also be captured from the pulse wave between any feature points (e.g., the trough points illustrated in FIG. 3A) appearing repeatedly on adjacent pulse waves. Accordingly, the method of capturing and calculating the second information in the present embodiment is much simpler than that of the related art, and is not limited by the position of the diastolic wave peak.

Besides, in comparison with the content of the related art as illustrated in FIG. 1, in the present embodiment, the physiological condition index may not only be calculated and obtained through the first and the second information obtained according to the time difference T1 between the foot point P1 and the pulse peak P2 and the time difference between the two foot points P1 and P1′. The physiological condition index may also be calculated and obtained through the first and the second information obtained according to the integrated area of the pulse wave between the foot point P1 and the pulse peak P2 with respect to the time axis and the integrated area of the pulse wave between the two foot points P1 and P1′ with respect to the time axis. The aforementioned two means for obtaining the first and the second information and the physiological condition index may be mutually compared and referenced for determining the blood circulation condition in the human body more accurately.

TABLE 1

| | Group 1 | Group 2 | Group 3 |
|---|---|---|---|
| Area A2/ | 4.44 ± 0.75 | 3.90 ± 0.70 | 3.54 ± 0.68 |
| Area A1 | | | |
| Time difference T2/ | 8.02 ± 1.29 | 6.65 ± 1.14 | 5.84 ± 0.85 |
| Time difference T1 | | | |

For example, referring to FIG. 3A to FIG. 3C, Table 1 shows averaged ratios of the integrated areas A2 to the integrated areas A1 of the pulse waves with respect to the time axis for subjects of different experiment groups. In the calculation results shown in Table 1, Group 1 represents healthy young people, Group 2 represents healthy aged people, and Group 3 represents diabetic patients with well controlled blood sugar. Generally speaking, the blood perfusion condition in the vessel declines along with the increase of the age and the increase in the degree of artery stiffness caused by disease. According to results shown in Table 1, in the group in a good health condition (e.g., the healthy young people of Group 1), the subjects have a less degree of arterial stiffness, and thus, the detection result of the ratio of the integrated area A2 to the integrated area A1 has a greater value. Namely, the ratio the integrated area A2 of the pulse wave between the two foot points P1 and P1′ (i.e., a pulse of a complete period) with respect to the time axis to the integrated area A1 of the pulse wave between the foot point P1 and the pulse peak P2 with respect to the time axis is greater than those of other groups. Thus, for the subjects of Group 1 who are young and have no cardiovascular diseases, they have better blood perfusion and circulation conditions in the vessel in comparison with the subjects of other groups.

In addition, according to the calculation result of the time difference T1/time difference T2, it also shows that in the group with better health condition (e.g., Group 1 as described above) the value of the time difference T1/the time difference T2 is greater, namely, the ratio of the time difference T2 between the two foot points P1 and P1′ to the time difference between the foot point P1 and the pulse peak P2 is greater than those of other groups. The results show that the subjects of Group 1 have better blood perfusion and circulation conditions in the vessel.

In the present embodiment, a user of the physiological detection device 100 may obtain a corresponding physiological condition index according to the ratio of the integrated area A2 to the integrated area A1 or the ratio of the time difference T2 to the time difference T1 and thereby, assesses the blood perfusion status in the human vessel and overall body circulation system functions.

Referring to FIG. 2 again, the physiological detection device 100 of the present embodiment includes a display 150 configured to display the physiological condition index. In the present embodiment, the display 150 is, for example, a liquid crystal display (LCD) or an organic light-emitting diode (OLED) display. In addition, the physiological detection device 100 also includes a memory 170, which may be any kind of data storage device, e.g., a flash memory, for storing the detection signal S1 and the physiological condition index. Furthermore, the physiological detection device 100 may by further equipped with a transmitter 160 capable of using, for example, the Bluetooth, WiFi and universal serial bus (USB) communication to transmit the physiological condition index to a device, e.g., a smart phone, a tablet computer or a remote server, for displaying and recording values through the transmitter 160, which contributes to long-term health monitoring.

FIG. 4 is a flowchart illustrating a physiological detection method according to an embodiment of the invention. Referring to FIG. 2 and FIG. 4, the physiological detection method of the present embodiment may be substantially divided into steps as follows. First, a detection portion of a human body is detected by the detector 110 to obtain a detection signal S1 (step S201). Then, the detection signal S1 is processed by the signal processor 120 to output a digital physiological signal S2 (step S202). Thereafter, the digital physiological signal S2 is received by the calculation module 130 to obtain first information and second information related to feature points of the digital physiological signal S2, and then, the digital physiological signal S2 is normalized by the calculation module 130 using the normalization processor 132. Thereafter, a ratio of the second information and the first information related to the feature points of the digital physiological signal S2 is calculated by the calculation module 130 using the physiological condition index calculator 134, so as to obtain a physiological condition index (step S203).

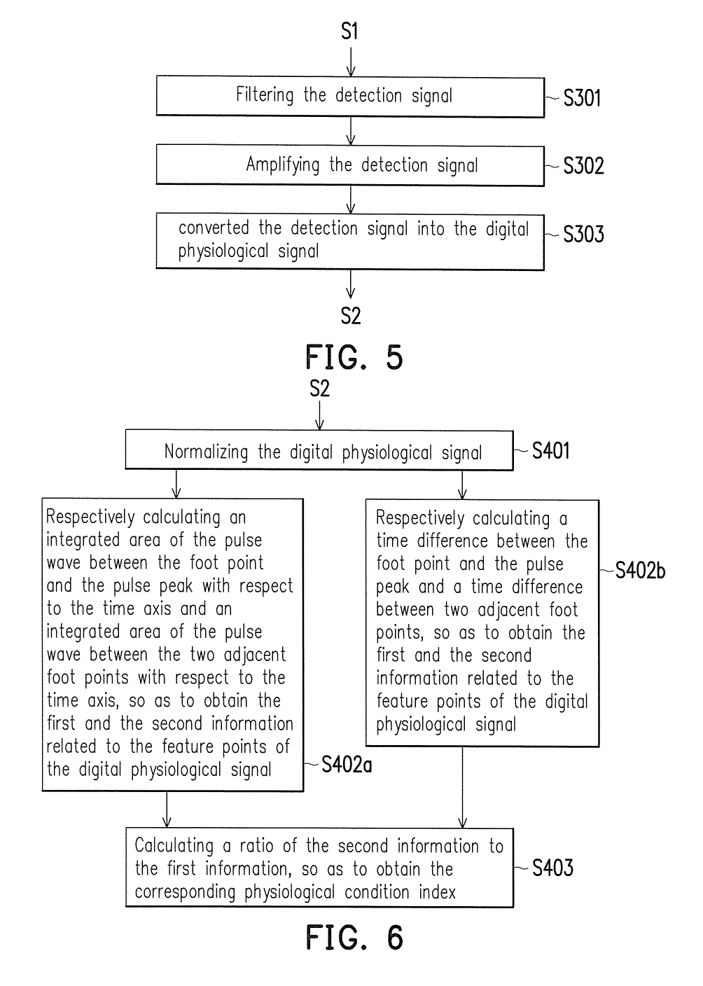

FIG. 5 is a flowchart of the signal processing step of the physiological detection method depicted in FIG. 4. Referring to FIG. 5 and FIG. 2, further to the above description, in the present embodiment, when the detection signal S1 is processed by the signal processor 120, the detection signal S1 is filtered by the signal processor 120 (step S301), and then, the detection signal S1 is amplified (step S302). Subsequently, the detection signal S1 which is originally an analog signal is converted into the digital physiological signal S2 by the analog-to-digital converter 126 (step S303). The sequence of the step of amplifying the detection signal S1 and the step of analog-to-digital conversion may be adaptively adjusted and changed according to the actual configuration of the signal processor 120 and demand for signal processing.

FIG. 6 is a flowchart of the step of calculating the physiological condition index of the physiological detection method depicted in FIG. 4. Referring to FIG. 4, FIG. 2 and FIG. 3A to FIG. 3C, in the present embodiment, the step of calculating the physiological condition index includes normalizing the digital physiological signal S2 by the normalization processor 132 of the calculation module 130 (step S401). Then, an integrated area of the pulse wave between the foot point P1 and the pulse peak P2 with respect to the time axis and an integrated area of the pulse wave between the two adjacent foot points P1 and P1′ with respect to the time axis are respectively calculated by the calculation module 130 to obtain the first and the second information related to the feature points of the digital physiological signal S2 (step S402a). Additionally, in another embodiment, the calculation module 130 may also select to calculate a time difference between the foot point P1 and the pulse peak P2 and a time difference between the two adjacent foot points P1 and P1′ to obtain the first and the second information related to the feature points of the digital physiological signal S2 (step S402b). Then, the ratio of the second information to the first information is calculated by the physiological condition index calculator 134 of the calculation module 130 to obtain the corresponding physiological condition index (step S403).

Based on the above, in the physiological detection method provided by the embodiments of the invention, the optical emitter of the physiological detection device emits the light, and then the light penetrating through the detection portion of the body or being reflected from the detection portion returns to the optical receiver of the physiological detection device to obtain the detection signal. Additionally, the detection signal is processed to obtain the digital physiological signal. In the physiological detection method of the invention, the ratio the integrated area of the pulse wave of the whole period with respect to the time axis to the integrated area of the pulse wave between the foot point and the pulse peak with respect to the time axis can be calculated according to the foot point and the pulse peak of each pulse wave of the digital physiological signal to obtain the corresponding physiological condition index. Moreover, In the physiological detection method of the invention, the ratio of the time difference between two foot points of adjacent pulse waves (which is the time of whole period) to the time difference between the foot point and the pulse peak can also be calculated to obtain the corresponding physiological condition index.

In the plurality of embodiments of the invention, when the pulse wave of the digital physiological signal of the subject does not have the diastolic wave, or the peak of the diastolic wave is unobvious, the physiological condition index of the subject can still be obtained through a simple calculation method. Furthermore, the user can assess the physiological condition, e.g., the blood perfusion and circulation condition in the vessel of the human body simply through the physiological condition index obtained by the physiological detection device and the method. Accordingly, the steps, time, and related testing equipment and cost required by a physiological detection process can further be reduced.

Although the invention has been disclosed by the above embodiments, they are not intended to limit the invention. It will be apparent to one of ordinary skill in the art that modifications and variations to the invention may be made without departing from the spirit and scope of the invention. Therefore, the scope of the invention will be defined by the appended claims.

What is claimed is:

1. A physiological detection method, comprising:
detecting a detection portion of a human body to obtain a detection signal;
processing the detection signal to output a digital physiological signal; and
receiving the digital physiological signal to calculate and obtain first information and second information related to a plurality of feature points of the digital physiological signal, and calculating a ratio of the second information to the first information to obtain a physiological condition index, wherein the digital physiological signal comprises a plurality of pulse waves generated according to a time sequence, and the feature points of the digital physiological signal include a pulse peak of each of the pulse waves and a foot point located at a forepart of a rising edge of each of the pulse waves.

2. The physiological detection method according to claim 1, wherein the first information is an integrated area of the pulse wave between the foot point and the pulse peak with respect to a time axis, and the second information is an integrated area of the pulse wave between two adjacent foot points with respect to the time axis.

3. The physiological detection method according to claim 1, wherein the first information is a time difference between the foot point and the pulse peak, and the second information is a time difference between two adjacent foot points.

4. The physiological detection method according to claim 1, wherein the step of processing the detection signal to output the digital physiological signal comprises:
filtering the detection signal;
amplifying the detection signal; and
converting the detection signal into the digital physiological signal.

5. The physiological detection method according to claim 1, wherein the step of calculating the information related to the feature points to obtain the physiological condition index comprises:
normalizing the digital physiological signal; and
calculating the physiological condition index according to the first information and the second information related to the feature points of the normalized digital physiological signal.

6. A physiological detection device, comprising:
a detector, adapted to detect a detection portion of a human body to obtain a detection signal;
a signal processor, receiving and processing the detection signal to output a digital physiological signal; and
a calculation module, receiving the digital physiological signal to calculate and obtain first information and second information related to a plurality of feature points of the digital physiological signal, and calculating a ratio of the second information to the first information to obtain a physiological condition index, wherein the digital physiological signal comprises a plurality of pulse waves generated according to a time sequence, and the feature points of the digital physiological signal include a pulse peak of each of the pulse waves and a foot point located at a forepart of a rising edge of each of the pulse waves.

7. The physiological detection device according to claim 6, wherein the first information is an integrated area of the pulse wave between the foot point and the pulse peak with respect to a time axis, and the second information is an integrated area of the pulse wave between two adjacent foot points with respect to the time axis.

8. The physiological detection device according to claim 6, wherein the first information is a time difference between the foot point and the pulse peak, and the second information is a time difference between two adjacent foot points.

9. The physiological detection device according to claim 6, wherein the detector is a photoplethysmograph (PPG) and comprises:
an optical emitter, configured to emit a light, wherein the light passes through the detection portion of the human body; and
an optical receiver, configured to receive the light passing through the detection portion to obtain the detection signal.

10. The physiological detection device according to claim 6, wherein the signal processor comprises:
a filter, configured to filter the detection signal;
an amplifier, configured to amplify the detection signal; and
an analog-to-digital converter, configured to convert the detection signal into the digital physiological signal.

11. The physiological detection device according to claim 6, wherein the calculation module comprises:
a normalization processor, configured to normalize the digital physiological signal; and
a physiological condition index calculator, configured to calculate the physiological condition index according to the information related to the feature points of the normalized digital physiological signal.
